(19) United States
(12) Patent Application Publication
(10) Pub. No.: US 2011/0021749 A1
(43) Pub. Date: Jan. 27, 2011

(54) Chemical Plugs used with Automated Organic Polymer Synthesizers

(76) Inventor: Thomas J. Demmitt, Fremont, CA (US)

Correspondence Address: SCHNECK & SCHNECK, P.O. BOX 2-E, SAN JOSE, CA, 95109-0005, US

(21) Appl. No.: 12/844,817

(22) Filed: Jul. 27, 2010

Related U.S. Application Data
(60) Provisional application No. 61/271,850, filed on Jul. 27, 2009.

(51) Int. Cl. C07K 1/04; C07H 21/00

(52) U.S. Cl. 530/334; 536/25.3

(57) ABSTRACT

A method and system for organic polymer synthesis utilizing flow through reaction vessels. A chemical plug is introduced selectively into reaction vessels that are inactive. Reactions continue in other reaction vessels. The chemical plug may be removed after reactions in the other reaction vessels have been completed. All reaction vessels are under a pressure differential which, with the use of the chemical plug, remains uniform.

### RELATED APPLICATIONS

The present application claims priority from provisional U.S. Patent Application No. 61/271,850 filed Jul. 27, 2009.

### TECHNICAL FIELD

The present invention relates to systems and methods for organic polymer synthesis and more specifically methods to

### BACKGROUND

Currently there are a number of systems for the production of organic polymers (incluind polypeptides and polynucleotides). These include chip based systems and systems using a vial. The systems using a vial generally employ a number of vials used in parallel. Contained within each vial is a solid support, to which the organic polymer is attached. Liquid reagents may then be introduced into the vial to add additional subunits onto the extending polymer chain.

A significant drawback of current parallel solid phase organic synthesizers is their limited ability to efficiently synthesize large numbers of target compounds (e.g. oligonucleotides, polypeptide) of various lengths in a same run. As noted in such a system, a plurality of flow through vials contain a solid support, a mechanism for reagent dispensing and a mechanism to drive liquid through the vials (e.g. a pump, vacuum sources, or other pressure differential generator). The organic polymers form on the solid support. In such parallel synthesis systems, the pressure differentials commonly vary along a synthesis run as individual molecule synthesis are completed because each resulting idle reaction vessel allows gas to pass at a higher flow than the vessels that contain molecules that are still synthesized. For example, even though some oligonucleotide or peptide sequences may be completed in some of the reaction vessels, an automated synthesis run continues until the longest sequence is completed. In such a situation some of the vials where the reaction is complete do not have a liquid contained in the vial. The gas flow rate through the completed reaction vessels causes a change in the pressure differential applied to the active reaction vessels. This in turn causes reagents to flow through the active reaction sites at a different rate.

Automated systems designed to maintain pressure differentials used to move reagents past reaction sites at optimal rates have been attempted with limited success. Previous attempts to compensate for the said change in pressure differentials in order to stabilize the pressure differential for a critical step in the synthesis include incrementally increase in push or drain time or addition of solvent wash to idle reaction wells prior to addition of the new building block into the still active wells. This increases consumption of gas or solvent, and generates extra waste while not fully eliminating the loss of backpressure experienced by the active reaction wells during part of the synthesis run.

A HTP (High Throughput) synthesizer is herein defined as a synthesis system that contains two or more wells or reaction sites that use pressure differential for moving reagents through the wells or sites in parallel. Wells may be in any orientation. Some common orientations are 96 well plate format, 384 well plate format, banks of wells in a circular pattern, and a single bank in a circular pattern (as shown in U.S. Pat. No. 7,192,558). As used herein, a reaction well is intended to mean a synthesis column, a reaction vessel or a space containing a solid support used for synthesizing the desired molecule. The present described methods and systems are applicable to different types of parallel solid phase synthesis of organic molecules including synthesis of DNA, modified DNA, RNA, modified RNA, polypeptides and other small molecules.

Current solid supports are made of loose controlled pore glass (CPG) beads or loose cross-linked or monolithic carbon-based polymer beads or are CPG- or polystyrene-embedded plugs. A synthesis run is made of finite number of synthesis cycle and the said synthesis cycle comprises the steps necessary to covalently attach one chemical building block to the molecule bound support. For instance, such cycle in solid phase oligonucleotide synthesis comprise the deblock, coupling, oxidation and capping steps with wash steps between one or more of these chemistry steps. In solid phase peptide synthesis, such cycle comprises the removal of a blocking group such as Fmoc or tBOC, coupling and capping steps with wash steps between one or more of these chemistry steps. During the synthesis process, wells are filled with reagent solutions according to their respective molecular sequence and according to their synthesis status. Chain elongations take place on the solid supports by reaction of molecular building blocks with a support bound molecule. With each molecular building block coupling, the molecular chain grows until all desired molecular building blocks have been added to the molecular chain. As used herein, a synthesis run ends after all reaction wells are idle, therefore all target molecules have been synthesized.

### SUMMARY

In an automated method for organic polymer synthesis, the method requires pushing reagents and draining wells by creating and controlling a pressure differential across the well from inlet side of the well to the outlet side of the well. As used herein, the said pressure differential across the well can be the result of applying a gas pressure or a vacuum or through the use of some type of a pump (e.g. a syringe pump, peristaltic pump, diaphragm pump, piston pump, gear pump, metering pump or other known pumps). Current pushing or draining procedures are performed on all or groups of wells simultaneously regardless of their synthesis status (i.e. active or idle status).

Wells with completed synthesis are left empty while subsequent chemical reactions are still performed on the still active (uncompleted) wells. As increasing number of molecules are completed, a resulting increasing number of idle wells affect the efficiency of pushing and draining steps during the plurality of reactions of each synthesis cycle which increasingly affect the quality of the synthesized molecules in the remaining active wells. It is an object to address this problem with a method and system that allows for more uniform pressure during synthesis.

### SUMMARY

The present disclosed embodiments improves current high throughput (HTP) syntheses by plugging idle reaction wells during a synthesis run with a flowable chemical plug (such as a highly viscous liquid) in order to maintain constant the pressure differential over an entire synthesis run. This in turn maintains a constant flow rate of reagents through the reaction sites which is needed to optimize the efficiency of each chemical reaction at each site while minimizing the consumption of gas, reagents and time.

### BRIEF DESCRIPTION OF THE DRAWINGS

FIG. 1 is a plan view of a first embodiment of a liquid plug device.

FIG. 2 is a plan view of a second embodiment of a liquid plug device.

### DETAILED DESCRIPTION

As used herein, a chemical plug is intended to mean a liquid or a blend of liquids that can be delivered to a well containing a completed target molecule in order to maintain a uniform pressure in reaction vessels that are either plugged or unplugged. In a system in which all flow reaction vessels are exposed to a common pressure differential, a uniform differential pressure is considered that pressure for each flow through reaction vessel in which all reaction vessels contain a liquid. This prevent a notable localized change in pressure that would occur if one or more of the reaction vessels did not contain a liquid or a plugging solid, such that gas could freely pass through that unplugged flow through reaction vessel.

The plug is not required to prevent all flow. However, the plug is not required to completely plug the flow through reaction vessel. Instead, the plug must simply provide for uniform pressure within the reaction vessels of a system which are subjected to a source of a pressure differential. To achieve this, the chemical plug is more viscous than a wash fluid used in the synthesis process to remove a reagent after each step in the process of forming the organic polymer. Some of the chemical plug may be forced by the pressure differential from the end of the reaction vessel. This would be collected from an open end of the reaction vessel and then taken to waste, also with the reagents and wash liquids removed from the reaction vessel after a reaction step.

An embodiment of a method for synthesizing organic polymers include the following steps:
a) providing viscous liquids that act as chemical plugs and are not harmful to the support bound molecules and introducing the chemical plug into a well in which the synthesis process is to be interrupted (e.g. the organic polymer synthesis is complete for this reaction or the;
b) introducing the said viscous liquid into a flow through reaction vessel to provide a chemical plug; and
c) removing the chemical plug as by washing of the viscous liquid out of the said reaction well at the end of a synthesis run or at any appropriate time in the synthesis run.

The chemical plug may provide a substantially airtight seal in the well at the pressures used for pushing and draining reagents used for synthesis. The chemical plug may be removed, for example by heating or by washing out the chemical plug with a wash solvent such as acetonitrile or other appropriate solvent. This was solution must be not harmful to the synthesized molecules. In some embodiments the air-tight seal provided by the chemical plug is retained (at lease in part) in the reaction vessel for a minimum of two synthesis cycles provided that it is not being drained by the succession of push and drain steps. Preferentially, the air-tight seal provided by the said chemical plug is permanent until a final wash step is performed at the end of the synthesis run or at any appropriate time of the synthesis cycle. A final wash step can also be a final plate wise detritylation or de-Fmoc or de-tBOC in oligonucleotide or peptide syntheses, respectively. Any solution that is compatible with the synthetic chemistry being performed and is capable of dissolving the chemical plug may be used.

In one embodiment, a system includes software allowing a user to set an initial delivery volume of the liquid plug to wells that are finished synthesis and then to add a replenishment volume at present intervals (intervals of at least two cycles). The volumes and number of cycles can be set by the user and will be set based on the flow of the liquid plug through the columns and the column volume. The factors that effect how often one needs to replenish the liquid plug are:
1) The flow characteristics of the columns being use.
2) The Flow Characteristics of the chemical Plug Material.
3) The pressure being used to empty the currently running wells.
4) The temperature of the environment if the liquid plug material is subject to viscosity changes based on temperature.

The chemical plug is selected to be able to flow into the wells that are to be interrupted. The chemical plug preferably will stay in the reaction vessel under normal pressure and temperature of synthesis for at least two and preferably at least several synthesis cycles before it needs to be replenished. One implementation uses as a chemical plug a substance that is solid at room temperature but flows at an elevated temperature. A heater in the reservoir containing that chemical makes the chemical liquid so it can be flowed into the idle reaction vessels. A heater on the device containing the wells can be used to make the chemical flow again so it can be washed out with a solvent that will not harm the product that was synthesized.

In an alternative embodiment is a chemical that is liquid at room temperature and can flow at room temperature into the reaction vessels that are to be interrupted. To be retained within these reaction vessels for multiple cycles this substance will need to be replenished at some interval and that interval is dependent on the flow rate of that chemical through the idle wells at room temperature and under the pressure being used for emptying wells that are active. This chemical must be able to be removed using a solvent that will not harm the product being synthesized. The chemical has a greater viscosity than the wash liquids, allowing the substance to be retained within the reaction vessel.

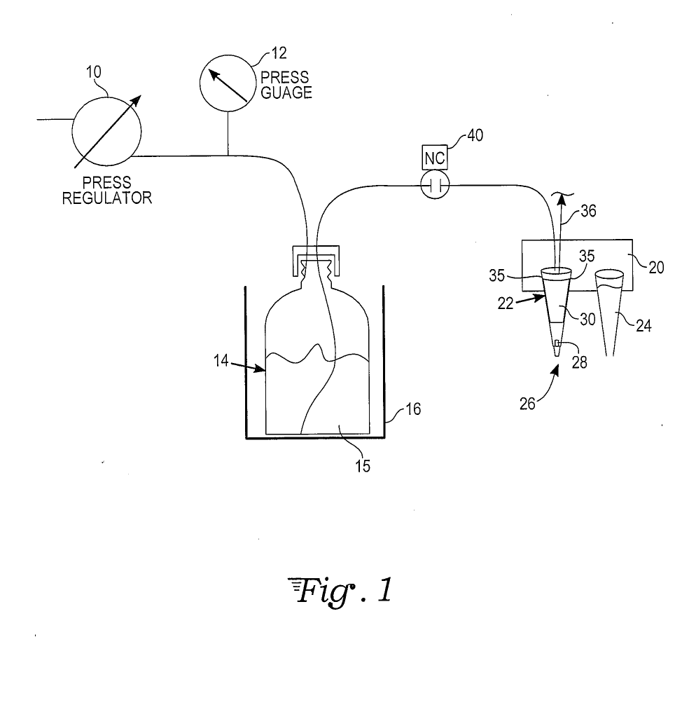

With reference to FIG. 1, a system for implementing the stated method includes a pressure regulator 10 that regulates the pressure from a gas source. A pressure gauge 12 allows monitoring of the pressure and cooperates with the regulator to control the gas pressure. The pressurized gas is introduced into a vessel 14 containing a chemical plug material 15. The vessel 14 may be placed in a heating element 16. If the chemical plug is solid at room temperature, the heating element allows the chemical plug to be heated into its liquid form. A valve 40 is used to control flow of liquid from the vessel 14 into the first reaction vessel 22. The pressure regulator 10 provides a pressurized head of gas over chemical plug substance 15. When valve 40 is opened, liquid from the vessel 14 will flow into a first reaction vessel 22.

The first reaction vessel 22 then contains a chemical plug that can solidify into chemical plug 30. The solid support 28 holds an organic polymer that has been synthesized. When the user desires to remove the chemical plug, a heater 35 may be employed to heat the contents of first reaction vessel 22. The liquid can then be removed by pressure out end 26. Alternatively or in addition to the pressure a wash solution can be delivered through tube 36 to make the chemical plug less viscous and drive the chemical plug from first reaction vessel 22. In an alternative, the heater 16 for the vessel containing the chemical plug material and the heater 35 for heating the first reaction vessel are not included. In this embodiment the liquid used as the chemical plug is viscous (more viscous than the wash solution) but is not solid at room temperature. This chemical plug will need to be replenished after a selected number of cycles of the synthesis reaction.

The first reaction vessel 22 and a second reaction vessel 24 are both contained in a housing 20 which has a pressure differential source, such as a vacuum or a pump. This allows a pressure to be applied such that reagents are driven through an open end of reaction vessels when a reaction of the reagents and a target on a solid substrate is complete. Both first vessel 22 and second vessel 24 are subject to the same pressure differential source. By plugging first reaction vessel 22, the pressure on second reaction vessel 24 is much more uniform.

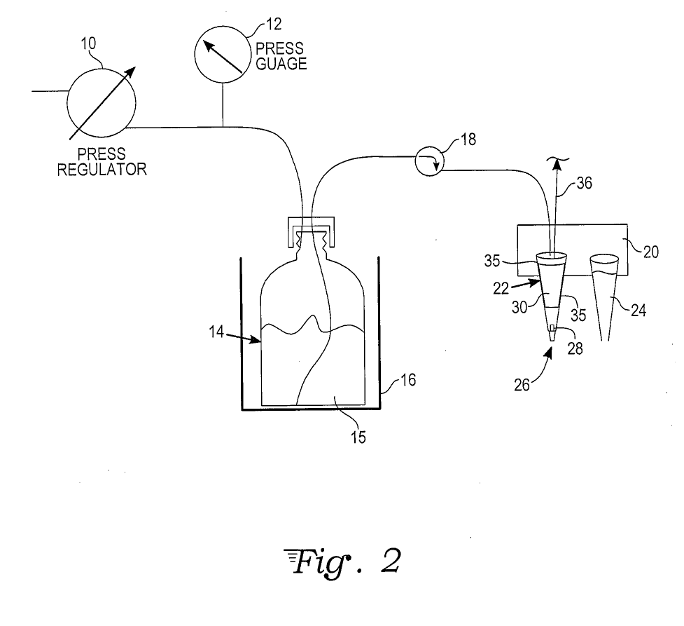

FIG. 2 discloses a similar system. In this system, the valve 40 is replace with a pump 18. Pump 18 can be used to pump the chemical plug liquid 15 from vessel 14 and into first reaction vessel 22.

The components of FIGS. 1 and 2 may be added onto any organic polymer synthesis system that uses multiple, flow through reaction vessels. Such a system is shown in U.S. Pat. Nos. 7,192,558 and 6,867,050, hereby incorporated by reference for all purposes herein. The systems for supplying the reagents to the reaction vessels has not been shown.

The disclosed embodiments rely on some or all of the properties of a chemical plug including melting point, boiling point, dynamic viscosity, surface tension and molecular weight. The reagent dispensing station is configured to house one liquid reservoir containing a chemical plug such that can be delivered to the idle flow through reaction vessels and one reservoir containing a washing solvent that will remove the chemical plug while not harming the target molecule.

The chemical plug, as noted may be a liquid or a blend of two or more liquids having a dynamic viscosity greater than the primary wash solvent used in the synthesis (such as acetonitrile in oligonucleotide synthesis or DMF in peptide synthesis). Preferentially, such liquids are chosen from, for example, benzonitrile, DMSO, ethylene glycol, glycerol. Application of a highly viscous, high boiling point liquid such as glycerol is preferred as evaporation of the reagent in the open environment is limited and its high viscosity substantially reduces the need of refilling idle wells during the synthesis run. Alternatively the chemical plug is may be a polymeric species such as polyethylene glycol, silicone oil or dewaxed oil.

We claim:

1. A method for the automated synthesis of organic polymers comprising:
a) providing a solid support contained with each of a plurality of flow through reaction vessels;
b) repeating a set of reactions in a plurality of said reaction vessels by sequentially introducing reagents into reaction vessels and removing said reaction reagents from said reaction vessel by a pressure driven migration of said reagents,
c) identifying at least one reaction vessel in which said reaction is desired to be temporarily interrupted;
d) introducing a chemical plug into said at least one reaction vessel in which said reaction is desired to be temporarily interrupted;
e) continue steps b and c in a plurality of reaction vessels which have not been plugged with the chemical plug;
f) removing the chemical plug from said at least one reaction vessel;
wherein, during steps b through e, said flow through reaction vessels are under a differential pressure, wherein said chemical plug results in an essentially uniform flow rate from reaction vessels not having said chemical plug.

2. The method of claim 1, wherein said chemical plug is a melted liquid that becomes solidified in the reaction vessel, wherein removing the chemical plug includes heating the chemical plug to liquefy the chemical plug and applying to the liquid chemical plug a pressure differential sufficient to migrate the chemical plug from the reaction vessel.

3. The method of claim 1, wherein the chemical plug is a liquid having a dynamic viscosity greater than the dynamic viscosity of a wash solvent used to remove reaction reagents.

4. The method of claim 1, wherein the chemical plug is a viscous liquid selected from the group consisting of benzonitrile, dimethyl sulfoxide, ethylene glycol and glycerol.

5. The method of claim 1, wherein the chemical plug is a polymeric liquid.

6. The method of claim 5, wherein the polymeric liquid is selected from a group consisting of polyethylene glycol, silicone oil, or a dewaxed oil.

7. The method of claim 1, wherein said organic polymers are either a polypeptide or a nucleic acid.

8. The method of claim 1, wherein said solid support is a controlled pore glass substrate.

9. The method of claim 1, in which step c includes identifying a reaction vessel in which formation of an organic polymer is complete.
